(19) United States
(12) Patent Application Publication
(10) Pub. No.: US 2023/0414363 A1
(43) Pub. Date: Dec. 28, 2023

(54) HEART VALVE PROSTHETIC RING SYSTEM

(71) Applicant: Nikola Dobrilovic, Chicago, IL (US)

(72) Inventor: Nikola Dobrilovic, Chicago, IL (US)

(21) Appl. No.: 18/463,230

(22) Filed: Sep. 7, 2023

Related U.S. Application Data
(63) Continuation of application No. 16/881,579, filed on May 22, 2020.
Application No. 16/881,579 is a continuation of application No. 16/251,965, filed on Jan. 18, 2019, now Pat. No. 10,695,180.
Application No. 16/251,965 is a division of application No. 14/051,787, filed on Oct. 11, 2013, now Pat. No. 10,182,913.
Application No. 14/051,787 is a continuation-in-part of application No. 13/871,327, filed on Apr. 26, 2013, now Pat. No. 9,839,516.
Application No. 14/051,787 is a continuation-in-part of application No. 13/658,050, filed on Oct. 23, 2012, now Pat. No. 9,889,009.
(60) Provisional application No. 61/639,482, filed on Apr. 27, 2012.
(60) Provisional application No. 61/641,338, filed on May 2, 2012.
(60) Provisional application No. 61/555,002, filed on Nov. 3, 2011.
(60) Provisional application No. 61/551,728, filed on Oct. 26, 2011.
(60) Provisional application No. 61/713,115, filed on Oct. 12, 2012.

(51) Int. Cl. A61F 2/24; A61B 17/04

(52) U.S. Cl. CPC A61F 2/2496; A61B 17/0483; A61B 2017/00783

(57) ABSTRACT

A heart valve prosthetic ring assembly includes an outer ring, and an inner ring configured and arranged to couple to the outer ring. The outer ring and inner ring having a pair of complementary mating surfaces configured and arranged to grip sutures therebetween when coupled together. Once coupled around sutures, the assembled ring set may be tightened down against the heart valve annulus to test the fit. If there are fit issues, the surgeon can remove the current ring set and replace it with another ring set of a different size/shape to determine a better fit. Once a proper size is determined, the surgeon may utilize the selected paired ring set as the final prosthetic reinforcing ring. The paired ring set may be secured in place with suture clasps which secure the ring sutures in place relative to the annulus and the ring set.

### CROSS-REFERENCE TO RELATED APPLICATIONS

This application is a continuation of U.S. application Ser. No. 16/881,579, filed May 22, 2020, which is a continuation of U.S. application Ser. No. 16/251,965 filed Jan. 18, 2019, now U.S. patent Ser. No. 10/695,180 issued Jun. 30, 2020, which is a divisional of U.S. application Ser. No. 14/051,787 filed Oct. 11, 2013 now U.S. patent Ser. No. 10/182,913 issued Jan. 22, 2019 which is a continuation-in-part of U.S. application Ser. No. 13/871,327 filed Apr. 26, 2013, now U.S. Pat. No. 9,839,516 issued Dec. 12, 2017, which claims the benefit of U.S. Provisional Application No. 61/641,338 filed May 2, 2012 and which further claims the benefit of U.S. Provisional Application No. 61/639,482 filed Apr. 22, 2012.

U.S. application Ser. No. 14/051,787 is also a continuation-in-part of US Application No. 13658050 filed Oct. 23, 2012 now U.S. Pat. No. 9,889,009 issued Feb. 13, 2018, which claims the benefit of U.S. Provisional Application No. 61/555,002 filed Nov. 3, 2011, and which further claims the benefit of U.S. Provisional Application No. 61/551,728, filed Oct. 26, 2011.

U.S. application Ser. No. 14/051,7881 also claims the benefit of U.S. Provisional Application No. 61/713,115 filed Oct. 12, 2012.

The entire contents of all claimed priority applications are incorporated herein by reference.

### BACKGROUND

### 1. Technical Field

The present disclosure relates generally to heart valve repair in open heart surgery and more specifically to a heart valve sizing ring system, a method to properly size a heart valve reinforcement ring prosthesis for proper implantation, and a prosthetic reinforcement ring system.

### 2. Background of the Related Art

Accurate selection of ring size and shape is a critical component of heart valve repair surgery. Current “annulus-sizers” or “valve-sizers” are, by design, not very accurate at actually assessing optimal size (they only provide an estimate) and, certainly, provide no ability for physiologic assessment of the repair procedure in terms of valve function after repair.

Therefore, there is a need in the industry for a prosthetic heart valve ring system that allows the surgeon to accurately assess the size of the ring prosthesis needed, allows assessment of the fit and function on the patient, i.e. whether the fit is leaky or too tight, and thereafter to utilize the ring system as the final reinforcing prosthetic.

### SUMMARY OF THE DISCLOSURE

The present invention solves the problems of the prior art by providing a prosthetic ring assembly that includes an outer ring with small gap formed therethrough, and having an inner mating surface on the interior surface of the ring. The ring assembly further includes an inner ring that is sized to snap fit into the outer ring and includes a reciprocal outer mating surface designed to engage the inner mating surface of the outer ring and firmly snap together yet prevent damage to sutures which are to be captured therebetween. Sutures may be captured between the rings to allow the surgeon to test the fit of the ring assembly. Once sure of the fit, the ring assembly may be permanently secured into position by various securing means including suturing and other mechanical suture tying methods.

The reciprocal surfaces preferably include outwardly deflected portions to direct sutures outward and away from the heart valve.

The inner and outer rings form a paired ring prosthetic assembly and a system which can include a plurality of paired ring sets of different sizes and shapes to both properly size the ring prosthetic in situ during surgery and which can be used as the final ring prosthetic for permanent placement.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 is a perspective view of an embodiment of a heart valve ring prosthetic assembly of the present invention;

FIG. 2 is an exploded view of an embodiment of a heart valve ring prosthetic of the present invention;

FIG. 3 is a cross-section view through line 3-3 of FIG. 2;

FIG. 4 is a perspective view of the sutures being gathered within the outer ring an embodiment of a heart valve ring sizing tool of the present invention;

FIG. 5 is a perspective view showing of an embodiment of a heart valve ring sizing tool of the present invention in place over a heart valve with the sutures trapped between the outer and inner rings;

FIG. 6 is a cross-section view through line 6-6 of FIG. 5;

FIG. 7 is a perspective view of an alternative embodiment where the inner ring includes the gap for corralling the sutures;

FIG. 8 shows an enlarged view of another embodiment of the heart valve prosthetic including suture engaging features (tie downs) on both the inner and outer rings; and

FIG. 9 is an enlarged view of yet another embodiment wherein the sutures include integrated tie clasps to permanently secure the prosthetic ring system in place.

### DESCRIPTION OF THE PREFERRED EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

The present invention is described generally for mitral heart valve repair surgery. But it is to be understood that the present invention may be adapted for use on tricuspid valve repair and aortic root/annulus remodeling procedures (such as Tirone David type-operations) where the present invention may be used to simulate down-sizing the aortic root as would be created by tube graft material.

The proposed device will: 1) provide a quick, more realistic, useful, functional, true-measurement of mitral annulus size, and 2) allow for critical physiologic assessment of valve function (and consequently the repair quality) prior to final and permanent ring selection and allow for such physiologic measurement and assessment using several different ring sizes and/or different ring models and shapes.

The key to proper/improved ring selection is not “just guessing” after estimation of size and shape based on examination/measurement of visualized anatomy but on the potential information gained from assessment of actual physiologic consequences secondary to ring implantation. The proposed prosthetic ring assembly will allow for a quick assessment of the valve annulus/repair with the ability to then again quickly assess the same repair with a different sized or shaped ring prosthetic prior to final selection.

A key feature of the present prosthetic ring assembly is that an evaluation of valve physiology (i.e. testing a particular size and shape ring on the valve) can be obtained prior to commitment to a particular ring, which, under normal circumstances, is then (permanently) sewn into position. Currently, it is only after a ring prosthetic is sewn into position, that the valve's repair status can be optimally assessed intraoperatively.

The principal design features a paired ring assembly which quickly “captures” and aligns the ring sutures after they have been placed in the mitral valve annulus and after any complex repair had been completed such as quadrangular resection, etc. The alignment/positioning of the ring sutures would be such that the annulus size and shape, corresponding to a particular ring size may be quickly and accurately assessed for the desired function of the new post-repair physiology, degree of leaflet coaptation, etc. The paired ring assembly may then be quickly exchanged for another ring assembly having a different size and/or shape. If several such measurements can be executed efficiently and safely, the surgeon will be much more informed and secure in his decision regarding proper ring selection as it will be based, at least to a significant degree, on actual measurement and more importantly on actual physiologic assessment.

It is the hope that such preliminary physiologic assessment will translate into the best final outcome for the patient. The added information provided by quick and more accurate assessment of valve function, as described above, has the potential to significantly diminish the dreaded consequence of having to entirely remove and replace a poorly functioning valve ring/repair after separation from bypass and realization that the valve repair is functioning sub-optimally (or that a sub-optimal repair is left in place and “accepted” even though the surgeon is clearly unhappy with the suboptimal result). “Undersizing” of the annulus can also be better avoided and thus reduce the incidence of the unfavorable complication of “SAM” (systolic anterior motion) or having suboptimal hemodynamics from a smaller valve orifice or ring prosthesis shape.

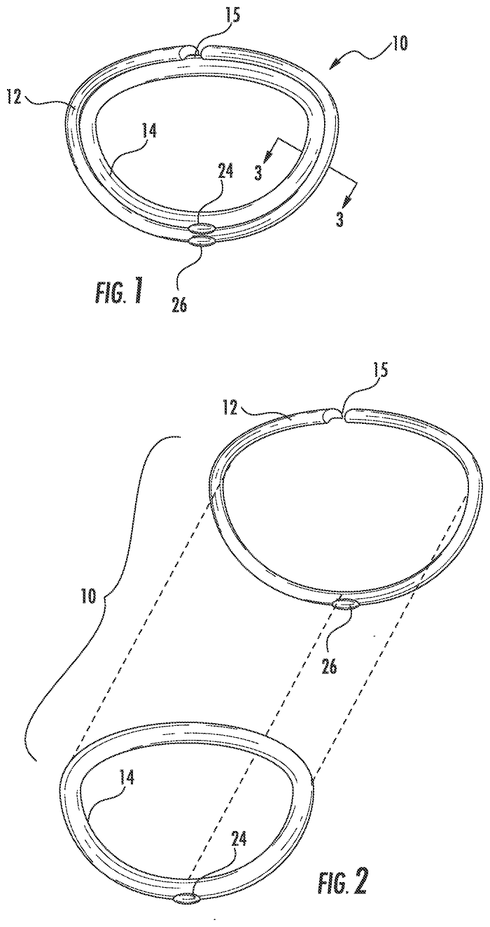

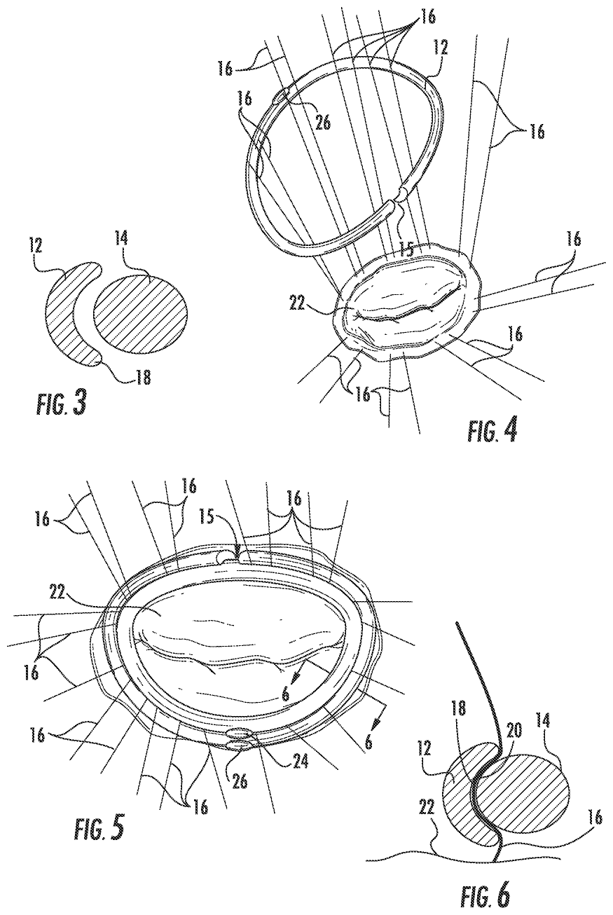

The described functions of this paired ring assembly and system can be achieved in a variety of ways. Referring now to FIGS. 1-2, a first embodiment 10 of the heart valve prosthetic ring assembly of the present invention includes two rings 12, 14 which “snap” together. The outer ring 12 may be an incomplete annulus, having a small gap 15, to allow it to be used to gather all of the annular sutures within the outer ring 12 in a quick and easy manner (as shown in FIG. 4). The inner ring 14 would be complete and would “snap” into the slightly larger outer ring 14. In doing so, the two rings 12, 14 would firmly, yet without damage to suture material, capture all sutures 16 in between the two rings 12, 14 (best seen in FIGS. 5 and 6). The device could then be “seated” into a final operational position (at annular level). The present prosthetic ring set is thus used for both sizing in situ and final permanent placement once the proper size is determined.

The inner ring 14 preferably includes a concave surface 18 on the outer portion of the ring 14. The outer ring 12 preferably includes a complimentary convex surface 20 on the inner portion of the ring 12. The concave and convex surfaces 18, 20 couple together to hold the rings 12, 14 in an assembled state as shown in FIG. 3. Because the inner ring 14 includes a concave surface 18 and the outer ring 12 includes a convex surface 20, the sutures 16, when captured between the rings 12,14, are deflected outwards and away from the heart valve 22 permitting assessment of the physiology by being able to better view the fit.

The inner ring 14 and outer ring 12 may each optionally include a tab 24, 26, respectively, extending from a portion of the ring 12, 14, preferably the bottom portion, to enable forceps to position and pull apart the two rings 12, 14. The tabs 24, 26 may be angled away from the center of the rings 12, 14 for easier gripping and manipulation.

Referring to FIG. 7, in a similar two-ring system, the prosthetic ring 200 includes an inner ring 214 with a gap 228, or defect, allowing sutures 16 to be gathered within the inner ring 214. The gap 228 also permits the inner ring 214 to flex inwardly. The inner ring 214 snap-fits into an outer ring 216. Together, the inner ring 214 and outer ring 216 may form a complete prosthesis. The inner ring 214 and outer ring 216 may include complimentary mating formations to permit a tight and secure fit together, yet permit the sutures 12 to be captured between the inner ring 214 and the outer ring 216. More specifically, the inner ring 214 may include a concave surface that complimentarily mates with a convex surface on the outer ring 216. The entire prosthesis 200 may then be tied down in place as is known in the art.

Because the embodiment includes an inner ring 214 and an outer ring 216 that can snap-fit around the sutures 16, the surgeon can determine an optimal fit for the prosthetic ring 200 prior to tying the prosthetic ring 200 in place around the annulus of the heart valve. The prosthetic ring 200 thus doubles as a sizer for determining the appropriate ring size for reinforcing the heart valve and then as the permanent prosthetic. The surgeon need not remove the prosthetic ring 200 once he has determined the fit is adequate. This prosthetic ring set 200 may be used for any heart valve including aortic root remodeling procedures as well.

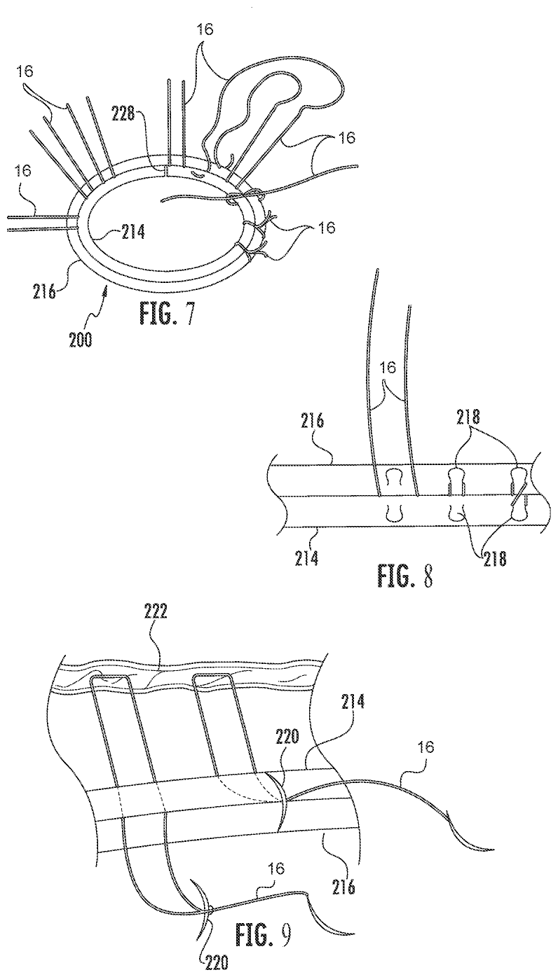

Referring to FIG. 8, the inner ring 214 and/or the outer ring 216 may also include notches or stays 218 formed on the surface facing away from the mating surfaces of either ring. These notches or stays 218 are configured to receive the suture 16 therein or tied thereto, respectively, and prevent the prosthetic ring 200 from shifting.

Referring to FIG. 9, the paired ring system may further includes a suture 16 with a self-tying knot and clasp 220 used to secure the assembled prosthetic ring 10/200 to the annulus 22/222 of the heart valve. Also, a suture tying hand tool may be used such as known in the art.

As described above, each particular paired ring set corresponds to a specified size and shape reproducing anticipated valve physiology should that particular ring size/shape be selected. The valve repair could be “tested” in standard fashion by “pressurizing” with saline, by examination, and/or by other techniques. When final ring function is reproduced in this manner, leaflet cooptation, as well as success of repair techniques, can be evaluated and compared at various ring sizes/shapes prior to final selection of the optimal, simply by swapping out the present paired ring set for another set of different size/shape. Several different ring sizes(/shapes) could be quickly swapped in and out for evaluation allowing for an informed, objective decision to be made. The paired ring sets may be made of a variety of materials. Standard considerations would of course apply such as cost, flexibility, rigidity, bio-reactivity, etc.

Ideally the paired ring sets would be composed of materials which would not harm/damage/weaken/fray sutures. One such example of the paired ring set would be two hard (metal, plastic, etc.) rings with a soft rubber-like outer layer on each. The metal, or other similar firm material, would provide support and accuracy in size and shape, while the outer rubber, or similar material with resilient properties, layer would not only protect, but also, delicately, yet firmly, “grasp” the sutures between the two rings. Ideally, the “grip” on the sutures would be such that paired ring set would not slip over the sutures passively but could actively be slid by the surgeon across the sutures to allow for seating into the desired position up against the valve annulus.

In some embodiments, the heart valve prosthetic ring set would be “stiff” to approximate the function of a “stiff” ring (possibly metal, plastic, etc). Though, other versions may exist to best approximate the physiologic support provided by various other types of rings (soft, partial, etc.).

In all embodiments, access for sutures through the outer ring can be in the form of a simple defect. Possibly one variant would provide for temporary exposure of a defect created by a quick “bending-apart” of the ring which would then “spring-back” into its original shape of what appears to be a complete ring (possibly facilitated by a mechanism to open the device). Alternatively, a small defect can simply remain uncovered, or a small latch can cover the defect, or the outer layer can slide over the defect.

Various mechanisms can be employed to hold the paired ring assembly (via the sutures) firmly up against the annulus. The simplest form may be simple friction provided by a rubber-like coating as described above. Other mechanisms/materials such as the self tying suture clasp 220 noted above may provide a similar function to secure the sutures firmly in place relative to the paired ring set.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

I claim:

1. A heart valve prosthetic ring assembly, comprising:
an outer ring; and
an inner ring configured and arranged to couple to the outer ring; and
the outer ring and inner ring having a pair of complementary mating surfaces configured and arranged to grip sutures therebetween when coupled together.

2. The heart valve prosthetic ring assembly of claim 1, wherein the outer ring further comprises a break in the outer ring.

3. The heart valve prosthetic ring assembly of claim 1, wherein the outer ring has a concave surface on an inner portion thereof and the inner ring has a convex surface on an outer portion thereof that snap-fit together.

4. The heart valve prosthetic ring assembly of claim 3, wherein the outer ring further comprises a break in the outer ring.

5. The heart valve prosthetic ring assembly of claim 1, further comprising a tab extending from the outer ring.

6. The heart valve prosthetic ring assembly of claim 5, wherein the tab is angled outwardly from the outer ring.

7. The heart valve prosthetic ring assembly of claim 1, further comprising a tab extending from the inner ring.

8. The heart valve prosthetic ring assembly of claim 5, further comprising a tab extending from the inner ring.

9. The heart valve prosthetic ring assembly of claim 1 further comprising a suture clasp for securing the sutures relative to the inner and outer rings.

10. A heart valve prosthetic ring system, comprising:
a plurality of paired ring prosthetic ring assemblies,
each of said plurality of paired ring prosthetic ring assemblies comprising and outer ring, and an inner ring configured and arranged to couple to the outer ring, and the outer ring and inner ring having a pair of complementary mating surfaces configured and arranged to grip sutures therebetween when coupled together,
each of said plurality of paired ring prosthetic ring assemblies having a different size wherein each of the plurality of paired ring prosthetic ring assemblies may be utilized for determining proper sizing and then permanently secured in place as a permanent implanted ring prosthetic.

11. The heart valve prosthetic ring system of claim 10, wherein each outer ring further comprises a break in the outer ring.

12. The heart valve prosthetic ring system of claim 10, wherein each outer ring has a concave surface on an inner portion thereof and each inner ring has a convex surface on an outer portion thereof that snap-fit together.

13. The heart valve prosthetic ring system of claim 12, wherein the outer ring further comprises a break in the outer ring.

14. The heart valve prosthetic ring system of claim 10, further comprising a tab extending from each outer ring.

15. The heart valve prosthetic ring system of claim 14, wherein the tab is angled outwardly from the outer ring.

16. The heart valve prosthetic ring system of claim 10, further comprising a tab extending from each inner ring.

17. The heart valve prosthetic ring system of claim 14, further comprising a tab extending from each inner ring.

18. The heart valve prosthetic ring system of claim 10 further comprising a suture clasp for securing the sutures relative to the inner and outer rings.
